(19) United States
(12) Patent Application Publication
(10) Pub. No.: US 2005/0033056 A1
(43) Pub. Date: Feb. 10, 2005

(54) Hydroxylated indole derivatives and uses thereof

(76) Inventor: Nancy Wong, N. Andover, MA (US)

Correspondence Address: CLARK & ELBING LLP, 101 FEDERAL STREET, BOSTON, MA, 02110, US

(21) Appl. No.: 10/498,751

(22) PCT Filed: Dec. 20, 2002
(86) PCT No.: PCT/US02/40992

Related U.S. Application Data
(60) Provisional application No. 60/344,214, filed on Dec. 21, 2001.

(51) Int. Cl. A61K 31/454; C07D 43/04

(52) U.S. Cl. 546/201; 514/323

(57) ABSTRACT

The invention provides hydroxylated indole derivatives and methods of using these derivatives to treat serotonergic diseases or conditions.

### FIELD OF THE INVENTION

This invention relates to hydroxylated indole derivatives and therapeutic methods employing these derivatives.

### BACKGROUND OF THE INVENTION

The neurotransmitter serotonin (5-hydroxytryptamine (5-HT)) is involved in regulating a broad range of physiological and behavioral activities. Abnormalities in serotonin levels, function, and metabolism have been shown to be associated with numerous diseases and conditions including, for example, psychiatric disorders (e.g., depression, anxiety, obsessive compulsive disorder, schizophrenia, aggression, and panic disorder), neurodegenerative diseases (e.g., Parkinson's disease and Alzheimer's disease), pain, migraine, headaches, obesity, and cardiovascular disorders (e.g., hypertension and unstable angina). Many approaches to treating these diseases and conditions thus involve modification of serotonin levels or activity.

Serotonin acts by binding to receptors that are present on cells in which it elicits its effects. Numerous serotonin receptors have been identified and cloned. These receptors have been divided into seven major classes, 5-hydroxytryptamine-1 (5HT₁) to 5-hydroxytryptamine-7 (5HT₇, on the basis of their primary structures and modes of interacting with transduction systems (Molecular Biology of 5HT Receptors, Neuropharmacol. 33:275, 1994). Serotonin receptor classes themselves are divided into subtypes. For example, subtypes 5HT_{1A}, 5HT_{1B} (formerly 5HT_{1Dbeta}), and 5HT_{1D} (formerly 5HT_{1Dalpha}) are known for the 5HT₁ receptor class (Trends in Pharmacol. Sciences 17:103, 1996). One approach to modifying the biological effects of serotonin involves blocking its binding to serotonin receptors. As an example of a drug having such an activity, N-methyl-[1-[1-(2-fluorophenethyl)piperidine-4-yl]-1H-indol-6-yl] acetamide (see, e.g., WO 98/43956 and EP 0 976 732 A1), which is currently under development for use as a central acting muscle relaxant, is a (5-HT) receptor antagonist. This drug potently binds to the 5-HT_{1A} and 5-HT₂ receptors with IC₅₀ in low nanomolar concentration range.

### SUMMARY OF THE INVENTION

The invention provides a compound of the formula: 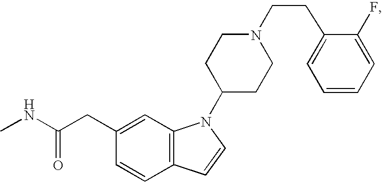
in which one or more methylene or methine proton is replaced with a hydroxyl group.

In one embodiment, the compound is of the formula: 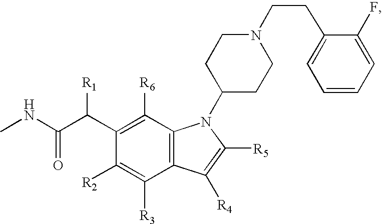
in which R₁, R₂, R₃, R₄, R₅, and R₆ are each independently a hydrogen atom or a hydroxyl group, provided that at least one of R₁, R₂, R₃, R₄, R₅, and R₆ is a hydroxyl group. In one example of such a compound, only one of R₂, R₃, and R₆ is a hydroxyl group, and R₁, R₄, and R₅ are hydrogen atoms. In another example, only one of R₂, R₃, and R₆ is a hydroxyl group, R₁ is a hydroxyl group, and R₄ and R₅ are hydrogen atoms.

In another embodiment, the compound is of the formula: 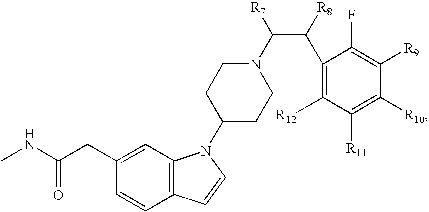
in which R₇, R₈, R₉, R₁₀, R₁₁, and R₁₂ are each independently a hydrogen atom or a hydroxyl group, provided that at least one of R₇, R₈, R₉, R₁₀, R₁₁, and R₁₂ is a hydroxyl group. In one example of such a compound, only one of R₇, R₈, R₉, R₁₀, R₁₁, and R₁₂ (e.g., R₈) is a hydroxyl group.

The invention also provides a compound of the formula:

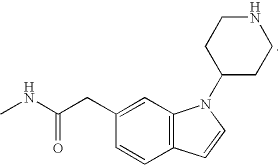

The invention also includes various isomers, such as diastereomers and enantiomers, salts, solvates, and polymorphs of the compounds described herein, as will readily be understood by those of skill in this art.

Also included in the invention are pharmaceutical compositions that include any of the compounds described herein, and pharmaceutically acceptable carriers or diluents, as well as methods of preventing or treating serotonergic diseases or conditions in subjects (e.g., humans), involving administration of such pharmaceutical compositions to the subjects. The invention also includes the use of the compounds described herein in preventing or treating serotonergic conditions, as well as the use of these compounds in the preparation of medicaments for these purposes.

Other features and advantages of the invention will be apparent from the following detailed description, the drawings, and the claims.

### BRIEF DESCRIPTION OF THE DRAWINGS

FIG. 1 is a series of graphs showing Compound 1 metabolic profiles in reconstituted in vitro human enzyme systems. The metabolic profiles were obtained by monitoring UV absorbance at 240 nm. The final protein concentration in the reaction mixtures containing the subcellular fractions was 2 mg/ml, and 50 pmol/ml in the mixtures containing recombinant proteins. The other reaction conditions are described below, in Experimental Methods.

FIG. 2 is a set of graphs showing Compound 1 MS (A) and MS/MS (B) spectra. The spectra were generated by an online MS/MS (API2000) after the compound was eluted from an HPLC column. The MS/MS product ion spectrum was generated after fragmentation of the quasi-molecular ions of Compound 1 (MH⁺) applying the collision energy. Details of the LC/MS and LC/MS/MS conditions are provided in Experimental Methods.

FIG. 3 is a set of graphs showing MS spectra of Compound 1 metabolites M1 (A) and M2 (B). The spectra were generated by an online Ion Trap MS (LCQ) after the metabolites were eluted from an HPLC column. The metabolites were formed in the reaction mixture containing HLM in the presence of NADPH, and the LC/MS conditions are described in Experimental Methods.

FIG. 4 is a set of graphs showing product ion spectra of metabolite M1 formed in a reaction mixture containing HLM: the M² spectrum by the ion trap MS (A), and the MS/MS spectrum by the MS/MS (B). The product ion spectra were generated after CID fragmentation of the quasi-molecular ions (MH⁺) of M1 eluted from an HPLC column. The in vitro metabolic reaction, LC/MS² (Ion Trap), and LC/MS/MS conditions are described in Experimental Methods.

FIG. 5 is a set of graphs showing product ion spectra of metabolite M2 formed in a reaction mixture containing HLM: the M² spectrum by the ion trap MS (A), and the MS/MS spectrum by the MS/MS (B). The product ion spectra were generated after CID fragmentation of the quasi-molecular ions (MH⁺) of M2 eluted from an HPLC column. The in vitro metabolic reaction, LC/MS² (Ion Trap), and LC/MS/MS conditions are described in Experimental Methods.

FIG. 6 is a set of graphs showing MS and MS² spectra of metabolite M₃ formed in a reaction mixture containing HLM: the MS spectrum (A), and the MS² spectrum (B). The spectra were generated by an online Ion Trap MS after M3 was eluted from an HPLC column. The MS² product ion spectrum was generated after CID fragmentation of the quasi-molecular ions (MH⁺) of M3. Details of the LC/MS (Ion Trap) conditions are provided in Experimental Methods.

FIG. 7 is a set of graphs showing MS and MS² spectra of metabolite M4 formed in a reaction mixture containing HLM: the MS spectrum (A), and the MS2 spectrum (B). The spectra were generated by an online Ion Trap MS after M4 was eluted from an HPLC column. The MS² product ion spectrum was generated after CID fragmentation of the quasi-molecular ions (MH⁺) of M4. Details of the LC/MS (Ion Trap) conditions are provided in Experimental Methods.

FIG. 8 shows proposed MS² fragmentation pathways of M4, as an example of the application of the nitrogen rule in LC/ESI-MS. The MS and MS² spectra are shown in FIG. 7, and the conditions for the generation of the spectra are described in Experimental Methods.

FIG. 9 shows proposed major CYP-mediated Compound 1 metabolic pathways in humans. The relatively minor metabolites, including the N-demethylated metabolite and possible monohydroxylated metabolites other than M1 and M2, are not included.

FIG. 10 is a set of Dixon plots showing Compound 1 inhibition of CYP2C19-mediated S-mephenyloin 4′-hydroxylation (A), and CYP2D6-mediated bufuralol 1′-hydroxylation (B). The recombinant enzymes were applied to obtain the single enzyme kinetics. Detailed experimental conditions are provided in Experimental Methods.

FIG. 11 shows the effect of Compound 1 on CYP expression in primary human hepatocytes after 72 hours of exposure. The protein expression was determined by Western immunoblotting, and polyclonal anti-human CYP antibodies and chemiluminescent reagents were used for the detection. Cells treated with TCDD (0.4 μM) served as the positive control for CYP1A induction, and cells treated with rifampicin (50 μM) served as the positive control for CYP3A, and probably CYP2C19, induction. Details of the experimental conditions are provided in Experimental Methods.

### DETAILED DESCRIPTION

The invention provides compounds that can be used in the treatment and prevention of diseases and conditions involving serotoninergic pathways. Several of these compounds are hydroxylated indole derivatives having the formula: 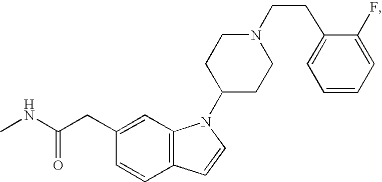
in which one or more methylene or methine proton is replaced with a hydroxyl group.

The hydroxyl group can be present, for example, in the indolyl acetamide portion of this structure, at any of positions R₁-R₆, as is indicated below: 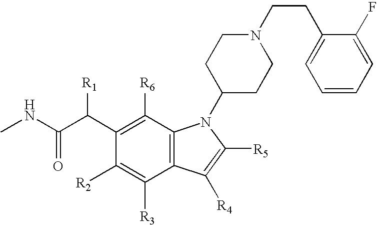
In one specific example, only one of R₂, R₃, and R₆ is a hydroxyl group, and R₁, R₄ and R₅ are hydrogen atoms. In another specific example, only one of R₂, R₃, and R₆ is a hydroxyl group, R₁ is a hydroxyl group, and R₄ and R₅ are hydrogen atoms.

Alternatively, the hydroxyl group can be present in the fluorophenethyl piperidine portion of the structure, at any of positions R₇-R₁₂, as is indicated below: 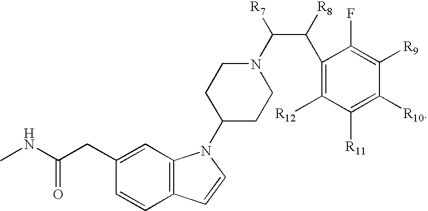
In one specific example, only one of R₇, R₈, R₉, R₁₀, R₁₁, and R₁₂ (e.g., R₈) is a hydroxyl group.

Another compound that is included in the invention, which also can be used in the treatment and prevention of serotonergic diseases and conditions, is of the following structure: 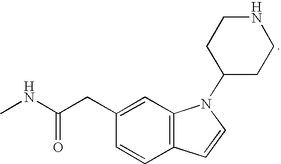
Synthesis

The compounds of the invention can be produced and isolated as described herein, or by the use of standard techniques that are known to those in the field of medicinal chemistry. For example, the compounds of the invention can be prepared directly from N-methyl-[1-[1-(2-fluorophenethyl)piperidine-4-yl]-1H-indol-6-yl] acetamide (1) (Tsukuba Research Laboratories of Eisai Co. Ltd.): 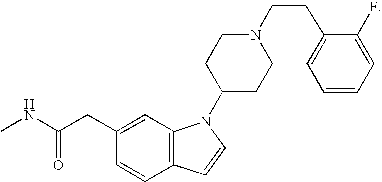
Selective hydroxylation of this compound is carried out by incubation of the compound in pooled human liver S9 fractions, in microsomal preparations, or with recombinant hepatic cytochrome P450 (CYP) forms (e.g., CYP1A2, CYP2C9, CYP2C19, CYP2D6, CYP2E1, or CYP3A4), in the presence of NADPH. Details of an example of this method are provided below, in Experimental Methods.

The compounds of the invention can be isolated and purified using standard methods in the art such as, for example, affinity chromatography, reverse phase, normal phase, cation exchange (e.g., of acid salts), or HPLC chromatography, or combinations of these methods. Selection of a suitable stationary phase, mobile phase, and solvent gradient is likely to depend on such variables as the lipophilicity, solubility, and variability in the retention times for each compound in the reaction mixture, and can readily be carried out by those of skill in this art.

Clinical Applications

The compounds of the present invention can be used in the treatment of any disease or condition that is associated with an abnormality in serotonergic neurotransmission. For example, the compounds can be used in the treatment of muscle spasm, hypertension, migraine, headache, cluster headache, anxiety, depression, dysthymia, panic disorder, obsessive-compulsive disorder, posttraumatic stress disorder, avoidant personality disorder, borderline personality disorder, phobia, a disorder of cognition, a memory disorder, a learning disorder, a neurodegenerative disease, anxiety and/or depression associated with senile dementia, Alzheimer's disease, or Parkinson's disease, cancer, cerebral infarct, a sexual disorder, dizziness, an eating disorder, pain, muscle spasm, chemical dependency or addiction, peptic ulcer, and attention deficit hyperactivity disorder. Additional examples of such diseases or conditions are known to those of skill in this art, and can likewise be treated or prevented using the compounds and methods of the invention.

The compounds of the invention can be administered to a subject using any route determined to be appropriate by one of skill in this art. For example, the compounds can be administered by use of oral, topical, parenteral, intravenous, intra-arterial, subcutaneous, intramuscular, intracranial, intraorbital, ophthalmic, intraventricular, intracapsular, intraspinal, intracistemal, intraperitoneal, intranasal, intrapulmonary, rectal, or other routes. Subjects that can be treated using the methods of the invention include, for example, humans, domestic pets, livestock, or other animals.

Conventional pharmaceutical practice can be employed to provide suitable formulations or compositions in which to administer the present compounds to subjects suffering (or at risk of suffering) from a serotonergic disorder or condition, and administration can begin before or after the subject is symptomatic.

Therapeutic formulations may be in the form of liquid solutions or suspensions, tablets or capsules (e.g., for oral administration), or powders, nasal drops, or aerosols (e.g., for intranasal formulations). Methods for making such formulations are well known in the art and are described, for example, in “Remington: The Science and Practice of Pharmacy” (20^{th} edition), ed. A. R. Gennaro, 2000, Lippincott Williams & Wilkins).

The compound can optionally be administered as a pharmaceutically acceptable salt. Examples include, but are not limited to, acetate, adipate, alginate, ascorbate, aspartate, benzoate, benzenesulfonate, bisulfate, borate, butyrate, camphorate, camphorsulfonate, carbonate, citrate, cyclopentanepropionate, digluconate, dodecylsulfate, ethanesulfonate, fumarate, glucoheptanoate, glutamate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, oxide, palmitate, pamoate, pectinate, persulfate, 3-phenylpropionate, phosphate, picrate, pivalate, propionate, salicylate, suberate, succinate, sulfate, tartrate, thiocyanate, tosylate, trifluoroacetate, undecanoate sulfate, bromide, chloride, fluoride, and iodide.

The formulations can be administered to subjects, such as human patients, in therapeutically effective amounts to provide therapy for a disease or condition associated with a serotonergic disorder or condition. Typical dose ranges are from about 0.1 μg/kg to about 100 mg/kg of body weight per day. An appropriate dosage of drug to be administered is likely to depend on variables such as the type and extent of the disorder, the overall health status of the particular subject, the formulation of the compound, and the route of administration. Standard clinical trials can be carried out to optimize the dose and dosing frequency for any particular compound.

Assays

Compounds of the present invention can be screened for serotonin (5-HT) receptor binding activity using any of a number of standard assays. For example, affinity for serotonin receptors can be measured using the methods described in JP98/01481 or JP-A-10-281752. In another example, a cell line expressing a human 5-HT receptor subtype can be employed in the affinity assay. Also see, e.g., Cheetham et al., Neuropharmacol. 32:737, 1993, and Leysen et al., Mol. Pharmacol. 21:301, 1982, for descriptions of additional methods that can be used.

Experimental Results

Summary

Compound 1, or N-methyl-[1-[1-(2-fluorophenethyl)piperidine-4-yl]-1H-indol-6-yl] acetamide, an antagonist of 5-hydroxytryptamine (5-HT) receptor subtypes 1A and 2, has activity in the treatment of skeletal muscle associated spasticity. We have characterized the in vitro metabolism of Compound 1 using human liver enzymes, including human liver microsomal preparations (HLM), human liver S9 fractions (HLS9), and individual forms of recombinant cytochromes P450 (CYPs). Our results show that Compound 1 is metabolized by CYPs to form monohydroxylated (M1 and M2), dihydroxylated (M3), and N-dealkylated (M4) metabolites. The structures of these major microsomal metabolites were obtained based on LC/MS/MS analyses. All of the four metabolites, M1-M4, were formed by CYP3A4. Metabolites M1, M2, and M4, were also formed by CYP2C19, and M2 and M4 were formed by CYP2D6. The potential CYP inhibition and induction of Compound 1 were also evaluated. Compound 1 was determined to be a competitive inhibitor of CYP2C19 and CYP2D6, with Kᵢ of 15 and 48 ELM, respectively, as determined by both Dixon plots and simultaneously nonlinear regression (SNLR) analyses. Induction of major CYP expression was not detected immunochemically after a 72 hour exposure to 10 or 50 μM of Compound 1 in primary hepatocyte cultures obtained from three subjects. These data show that Compound 1 is likely to metabolically interact with major human CYP enzymes, including CYP2C19, CYP2D6, and CYP3A4, and predicts a low risk of drug-drug interaction. The details of these experiments are provided below.

Materials and Experimental Methods

Chemicals

N-methyl-[1-[1-(2-fluorophenethyl)piperidine-4-yl]-1H-indol-6-yl] acetamide, was obtained from Tsukuba Research Laboratories of Eisai Co. Ltd. (Ibaraki, Japan). (+/−)-Bufuralol, (+/−)-l′-hydroxybufuralol, 6-hydroxychlorozoxazone, S-mephenyloin, 4′-hydroxy-S-mephenyloin, and monohydroxylated warfarin metabolites (6-, 7- and 10-hydroxywarfarin) were purchased from Gentest Corp. (Woburn, Mass.). Chlorzoxazone, coumarin, albendazole, R-propranolol, 4′-chlorowarfarin, rifampicin, NADPH, TRIZMA, magnesium chloride, potassium phosphates, and rac-warfarin were obtained from Sigma Chemical Corp. (St. Louis, Mo.). TCDD (2,3,7,8-tetrachlorodibenzo-p-dioxin) was purchased from NCI Chemical Carcinogen Reference Standard Repositories (MRI, Kansas City, Mo.). Optically pure R- and S-warfarin were prepared from the racemic mixture by the differential crystallization method (West et al., JACS 83:2676-2679, 1961). The purity of warfarin enantiomers was at least 98%, as determined by chiral HPLC, mass spectrometer, and NMR analyses. All solvents used for the HPLC analyses were HPLC grade.

Enzymes and Hepatocytes

Pooled human liver microsomal preparations and S9 fractions were purchased from Gentest Corp. (Woburn, Mass.). The insect microsomal preparations containing cDNA-expressed recombinant human CYPs and the control insect microsomal preparations were also purchased from Gentest Corp. (Woburn, Mass.). Primary cultures of human hepatocytes from three female Caucasian donors (age: 55, 56, and 79) and the culture media were purchased from In Vitro Technologies (Baltimore, Md.). These donors did not have recorded liver disease or damage. Two of the donors were smokers (age: 55 and 79).

Other Materials

Polyclonal goat anti-human CYP1A1/2 and anti-human CYP3A4 antibodies, as well as monoclonal mouse anti-human CYP2D6 antibodies, were obtained from Gentest Corp. (Woburn, Mass.). Polyclonal rabbit anti-human CYP2C19 antibodies were obtained from Diagnosis Research Laboratories (Flanders, N.J.). All secondary antibodies were obtained from Sigma Chemical Corp. (St. Louis, Mo.). The electrophoresis apparatus and accessories were obtained from BioRad (Hercules, Calif.) or Pierce (Rockford, Ill.). The antibiotics (streptomycin/penicillin) and buffers for the electrophoresis were purchased from Gibco BRL (Rockville, Md.) or BioRad (Hercules, Calif.).

Metabolite and Metabolic Enzyme Identification

The metabolism of Compound 1 was determined by the disappearance of Compound 1 or the appearance of Compound 1 metabolites in reaction mixtures as compared to respective controls. Compound 1 was incubated in the reconstituted in vitro reaction systems containing pooled human liver S9 fractions, microsomal preparations, or recombinant hepatic CYP forms, including CYP1A2, CYP2C9, CYP2C19, CYP2D6, CYP2E1, and CYP3A4. The reaction mixture (total volume of 250 μL) contained 0.5 mg of liver microsomal protein, 50 or 25 pmol of recombinant CYP, and Compound 1 (50-80 μM) in 50 mM Tris buffer containing 15 mM MgCl₂ (pH 7.4). After incubating at 37° C. with gentle shaking for 1 minute, the reaction was initiated by adding 25 μL of NADPH solution (20 mg/mL), and was carried out for 1 hour. The reaction was terminated by quickly cooling the test tube on ice, followed by the addition of an equal volume of 100% methanol. The samples were centrifuged in a desktop centrifuge at 14,000 rpm for 5 minutes, and the supernatants were filtered through syringe filters (13 mm, 0.45 μm). The filtrates were subjected to analysis using LC/MS (ion trap) or LC/MS/MS.

CYP Inhibition Assays

The incubations were carried out in test tubes (12.0×75 mm). The incubation mixtures (250 μL) contained 0.5 mg of HLM protein, 50 (CYP1A2 and CYP2E1) or 25 (CYP2C9, CYP2C19, CYP2D6 and CYP3A4) pmol of recombinant human CYPs, Compound 1 (10 or 50 μM), probe substrate (R—, S-warfarin, chlorzoxazone, S-mephenyloin, or bufuralol) at different concentrations, and 0.5 mg of NADPH in 50 mM Tris buffer containing 15 mM MgCl₂ (pH 7.4). Compound 1 was added to the reaction mixture 5 minutes prior to the addition of the probe substrate. After incubating in a 37° C. waterbath with gentle shaking for 1 minute, the reaction was initiated by adding 25 μl of NADPH solution (20 mg/mL), and was carried out for 15-60 minutes. The reaction mixture containing only the probe substrate was used as the control. After the incubation, the reaction mixture was extracted by mixing with 250 μL of methanol containing the appropriate IS. After vortex mixing and centrifuging in a desktop centrifuge at 14,000 rpm for 5 minutes, the supernatant was filtered through a syringe filter (13 mm, 0.45 μm) into an HPLC vial. The filtrate was subjected to analysis.

If inhibition was detected, the inhibitory potency was determined using recombinant CYPs. Compound 1 appeared to inhibit the activities of CYP2D6 and CYP2C19. Therefore, three concentrations of S-mephenyloin (30, 80, and 200 μM) and bufuralol (5, 10, and 40 μM), and a range of concentrations (0-200 μM) of Compound 1 were used for the construction of Dixon plots and simultaneously nonlinear regression (SNLR) analyses were performed. The incubation conditions and sample preparations were the same as previously described.

The quantification was based on the calibration curves, and the quality control (QC) samples were applied to ensure the quality of the experiments. Samples for the standard curves and QC were prepared in a similar manner to those of the reaction samples.

CYP Induction Assays

Hepatocyte Treatment and Sample Preparation

Upon receiving a primary culture of human hepatocytes in 6-well plates, the culture medium containing streptomycin/penicillin was refreshed (2 ml/well). After acclimatizing in 5% CO₂ and 37° C. overnight, the cells were treated with the vehicle (negative control), the prototypic CYP inducers, including TCDD (0.4 μM) for CYP1A and rifampicin (50 μM) for CYP3A and possibly CYP2C (Liu et al., Arch. Biochem. Biophys. 389:130-134, 2001; Xu et al., Chem. Biol. Interact. 124:173-189, 2000; Feng et al., Brit. J. Clin. Pharmacol. 45:27-29, 1998; Gerbal-Chaloin et al., Drug Metab. Dispos. 29:242-251, 2001), and Compound 1 (10 and 50 μM) for 72 hours. Compound 1 stock solution was added to the culture medium at a 1:400 ratio (v/v). The culture medium and testing compounds were replenished every 24 hours.

At the end of the treatment, the cells were harvested into 1 mL of phosphate-buffered saline (PBS) after washing twice. The cells were precipitated by centrifugation, and were resuspended in PBS (approximately 50 μL). The sample preparation, after mixing with 200 mL of Laemmli buffer (62.5 mM Tris-HCl containing 2% SDS, 25% glycerol, and 0.01% bromophenol, pH 6.8) and being rocked for 2-4 hours, was mixed with 10 μL of 2-mercaptoethanol, and heated at 90° C. for 10 minutes before electrophoresis.

Western Immunoblotting Analysis

Proteins were resolved in a 12% SDS-PAGE gel using a mini gel apparatus at a constant voltage (60 mV/gel) for 70-80 minutes, and transferred onto a polyvinylidene difluoride (PVDF) membrane using a membrane-transferring unit at a constant voltage (60 mV/membrane) for 60 minutes. The membrane was blocked by 5% non-fat dried milk (NFDM) blotting buffer (PBS containing 0.05% Tween 20) at 4° C. overnight. The membrane was rinsed with the blotting buffer, and probed by 1:1,000 diluted anti-human CYP antibodies in 2.5% NFDM blotting buffer for 1 hour at room temperature. The membrane was rinsed six times (10 minutes each time) with the blotting buffer, and exposed to 1:10,000 diluted secondary antibodies labeled with horseradish peroxidase for 1 hour at room temperature. After being extensively rinsed with the blotting buffer, the membrane was exposed to the substrate of peroxidase (enhanced chemiluminescence reagent). CYP proteins were detected by fluorescence using an X-ray developer.

Instrumentation

Identification of Metabolites and Enzymes

LC/MS/MS systems were applied for the metabolite identification. The operation conditions were described as follows.

1. LC/MS (LCQ ion trap mass spectrometer, Finnigan Corp., San Jose, Calif.): Hewlett Packard 1100 HPLC system (Waldbronn, Germany) consisted of a binary pump, an autosampler, a column compartment unit, and an online variable wavelength UV detector (VWD). The detector was monitored at 270 nm, which is the UVₘₐₓ of Compound 1 predetermined by scanning between 225-400 nm using a photodiode array detector (PDA). The metabolites were separated on a Hewlett Packard Eclipse C18 column (150×2.1 mm). The mobile phases were 10 nmM ammonium acetate at pH 4.8 (A) and acetonitrile (B). The gradient (B) was 10% (0-6 minutes), 22% (12-22 minutes), 90% (25-30 minutes), and 10% (31 minutes and after). The flow rate was 0.25 mL/minute. Software Navigator (Version 1.2, Finnigan Corp.) was used to control the HPLC and MS and to acquire the data. The MS was operated at positive electrospray ionization (ESI) with 5.2 kV ionization potential and 220° C. heated capillary temperature. The product ion spectra were generated under 24V collision energy, which was optimized for the fragmentation of Compound 1.

2. LC/MS/MS (SCIEX API2000 triple quadrupole mass spectrometer, PE Biosystem, Foster City, Calif.): The same HPLC system described above was coupled with a tandem mass spectrometer. The metabolites were separated on a Sulpelco Discovery C18 column (150×2.1 mm), and the mobile phase described previously was run at 1.0 mL/min with a 1:10 split. The gradient (B) was 20% (0-6 minutes), 60% (14-18 minutes), 95% (20-24 minutes), and 20% (25 minutes and after). The operation of the HPLC and the MS/MS was controlled by MacChrom (Version 1.6, PE Biosystem). The MS/MS was operated at positive ESI with 5.0 kV ionization potential and 400° C. ion source temperature. The product ion spectra were generated applying collision-induced dissociation (CID) with optimized ion optic parameter settings.

CYP Substrate Assays

1. CYP1A2/2C9/3A4-mediated warfarin 6-, 7-, 8-, and 10-hydroxylation (Zhang et al., Drug Metab. Dispos. 23:1339-1346, 1995; Rettie et al., Chem. Res. Toxicol. 5:54-59, 1992; Brian et al., Biochemistry 29:11280-11292, 1990): Hewlett Packard 1100 HPLC system (Waldbronn, Germany) comprised a binary pump, an autosampler, a column compartment unit, a photodiode array detector (PDA), and a fluorescence detector. The system was controlled by ChemStation (Version 6.03, Hewlett-Packard). Warfarin and the metabolites were resolved on a Hewlett Packard Zorbax ODS C18 column (250×4.6 mm). The flow rate was 1 mL/minute. The mobile phases were 250 mM ammonium acetate at pH 4.9 (A), and 100% acetonitrile (B). The gradient (B) was 10% (0 minutes), 40% (5-10 minutes), 60% (16-19 minutes), 90% (22-26 minutes), and 10% (27 minutes and after). The warfarin metabolites, 6-, 8-, and 10-hydroxywarfarin, were monitored by UV at 313 nm, and 7-hydroxywarfarin by fluorescence at Ex₃₂₀ nm/Em_{380 nm}.

2. CYP2E1-mediated chlorzoxazone 6-hydroxylation (Court et al., Biopharm. Drug Dispos. 18:213-226, 1997): The equipment was similar to the system described for warfarin hydroxylations, with the exception that a VWD instead of a PDA was used. The HPLC column and mobile phases were also identical to those used in the warfarin assay. The gradient (B) was 10% (0 minutes), 40% (6-14 minutes), 90% (16-20 minutes), and 10% (21 minutes and after). 6-hydroxychlorzoxazone was monitored by UV at 280 nm.

3. CYP2D6-mediated bufuralol 1′-hydroxylation (Boobis et al., Bioch. Pharmacol. 34:65-71, 1985): The HPLC system was interfaced with a SCIEX API2000 triple quadrupole mass spectrometer (PE Biosystem, Foster City, Calif.). A Sulpelco Discovery C18 column (150×2.1 mm) and isocratic mobile phase was applied. The mobile phase, 10 mM ammonium acetate-acetonitrile/60:40, was run at 0.2 mL/minute. The operation of the LC/MS/MS was controlled by use of software, MacChrom (Version 1.6). The MS/MS was operated at positive ESI with 5.5 kV ionization potential and 500° C. ion source temperature. Multiple reaction monitoring (MRM) was applied for the quantification. The MRM transition ions were m/z 278→186 for 1′-hydroxybufuralol, and m/z 266→234 for the internal standard (1S) albendazole.

4. CYP2C19-mediated S-mephenyloin 4′-hydroxylation (Goldstein et al., Biochemistry 33:1743-1752, 1994): The LC/MS/MS system, including the separation column, described for the bufuralol hydroxylation was used. The isocratic mobile phase, 100 mM formic acid-acetonitrile/75:25, was run at 0.25 ml/minutes. The operation of the LC/MS/MS was controlled by MacChrom (Version 1.6). The MS/MS was operated at positive ESI with a 5.5 kV ionization potential and 100° C. ion source temperature. MRM was applied for the quantification. The MRM transition ions were m/z 235→150 for 4′-hydroxymephenytoin, and m/z 260183 for the IS R-propranolol.

Data Analysis

Data were acquired and analyzed by ChemStation (Version 6.03, Hewlett Packard, Waldbronn, Germany) for 6-, 7-, and 10-hydroxywarfarin and 6-hydroxychlorzoxazone, and MacQuan (Version 1.6, PE Biosystem, Foster City, Calif.) for 1′-hydroxybufuralol and 4′-hydroxymephenytoin. Quantification was based on peak area ratios of metabolites over the respective IS against the respective concentration of the metabolites. The standard curves were generated by linear regression with (6-, 7-, and 10-hydroxywarfarin, 1′-hydroxybufuralol, and 4′-hydroxymephenytoin) or without (6-hydroxychlorzoxazone) a weighting factor (1/x²). The metabolic rates were determined using Excel (Microsoft Office 97, Microsoft Corporation, Redmond, Wash.) or SigmaPlot (Version 6.00, SPSS Inc. Chicago, Ill.). Apparent inhibition constants (Kᵢ) were estimated by Dixon plots generated by the linear regression analyses, and by simultaneously nonlinear regression (SNLR) analyses applying the reversible inhibition models of Michaelis-Menten kinetics. The equations of velocity or turnover rate derived from these models are as follows:
V=Vₘₐₓ/(1+Kₘ/S/(1+I/Kᵢ) (1)
V=Vₘₐₓ/(1+I/Kᵢ+Kₘ/S) (2)
V=Vₘₐₓ/(1+Kₛ/S)/(1+I/Kᵢ) (3)
V=Vₘₐₓ/((1+I/Kᵢ)+(1+Kₛ/S)(1+I/Kᵢ) (4)
Equation (1) is for the competitive inhibition model, Equation (2) is for the uncompetitive model, Equation (3) is for the noncompetitive model, and Equation (4) is for the mixed inhibition model. S is the substrate concentration, and I is the inhibitor concentration. Vₘₐₓ is the maximum turnover rate, and Kₘ is the substrate concentration at which the turnover rate is the half of the maximum. Kᵢ is the competitive inhibition constant, while Kᵢ′ is the uncompetitive inhibition constant. Ks is the dissociation constant of the enzyme-substrate complex. Statistical analyses were performed using SigmaPlot and SigmaStat (Version 2.03, SPSS Inc. Chicago, Ill.). PowerPoint (Microsoft Office 97, Microsoft Corporation, Redmond, Wash.) was applied for the reconstruction of the images of Western immunoblots after being scanned.
Results
Metabolite Identification

Several metabolites were detected in the reconstituted systems containing pooled HLM or HLS9 using mass spectrometers. The formations of these metabolites were NADPH-dependent, indicating the involvement of CYPs. The possible biotransformations were monooxidations at several positions, N-dealkylation at either the piperidinyl or methyl amido moiety, and multiple oxidations at different sites, such as sequential monooxidations to form diol metabolites.

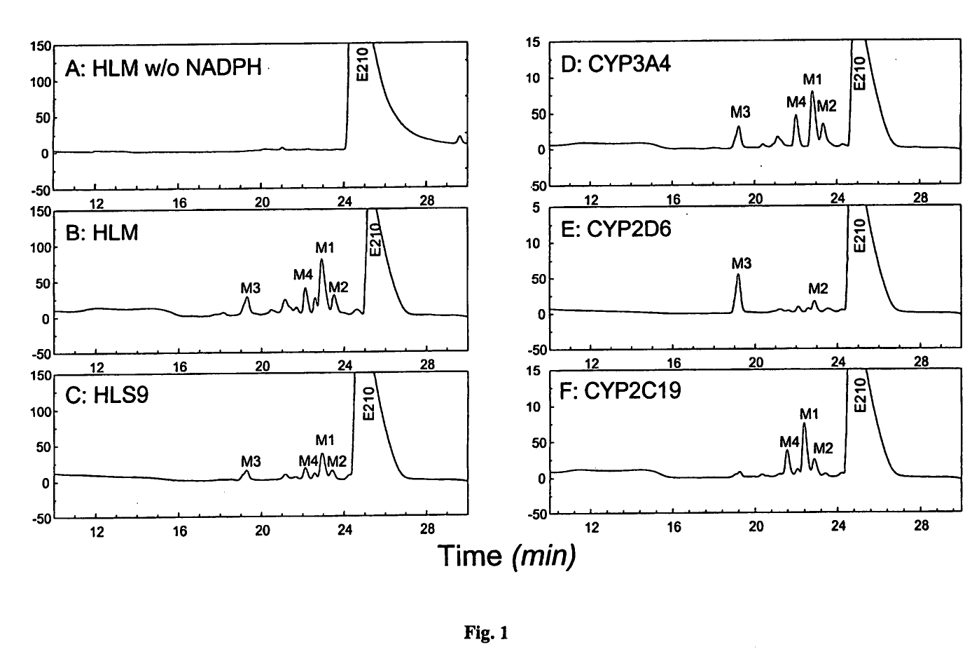

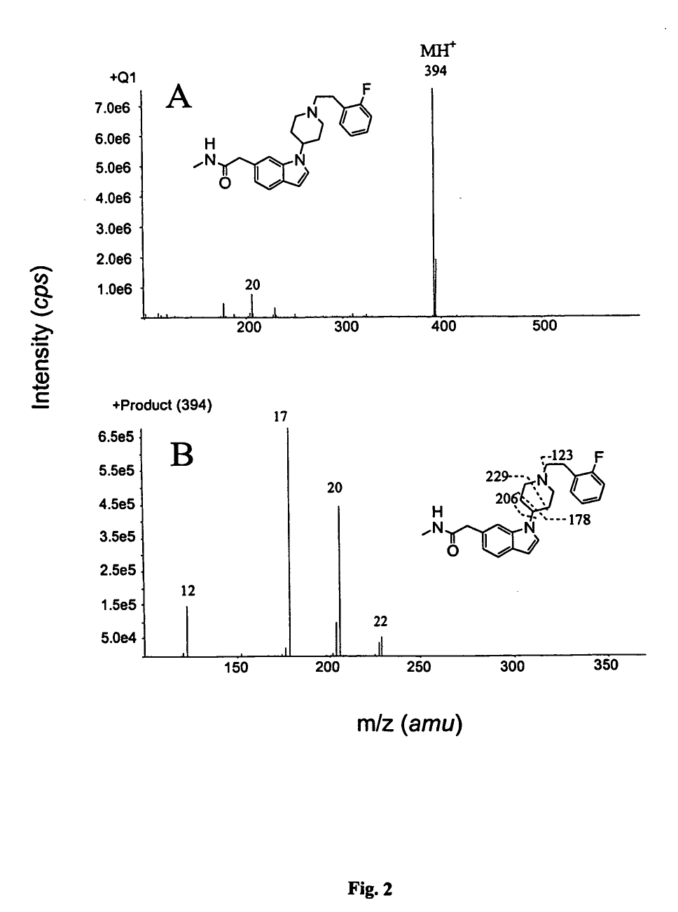

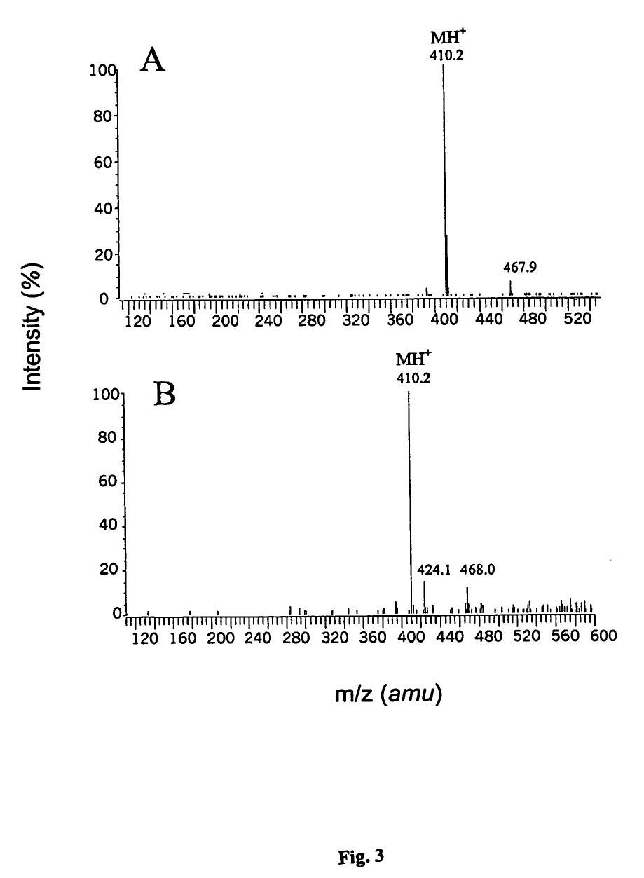

The identification of the major metabolites was undertaken using MS/MS spectral analyses. These metabolites (M1-4), listed in Table 1 (below), were detected online by both UV absorbance at 270 nm (FIG. 1) and MS total ion current (TIC) scanned between 60-1800 amu. The MS/MS fragmentation patterns of the phase 1 metabolites and parent compound are often similar. The MS spectrum and the MS/MS product ion spectrum of Compound 1 used as references for the spectral interpretation for the metabolites were first determined (FIG. 2). The predominant MS ion at m/z 394 was the MH⁺ ion of Compound 1, and its intensive MS/MS product ions at m/z 178, 206, 123, and 229 were likely formed after the CID fragmentations at the positions proposed in FIG. 2. Two of the major metabolites (M1 and M2) formed in reconstituted system containing HLM or HLS9 exhibited the MH⁺ ion at m/z 410 (FIG. 3). Apparently, these were the monohydroxylated metabolites because of 16 mass unit increments, compared to Compound 1.

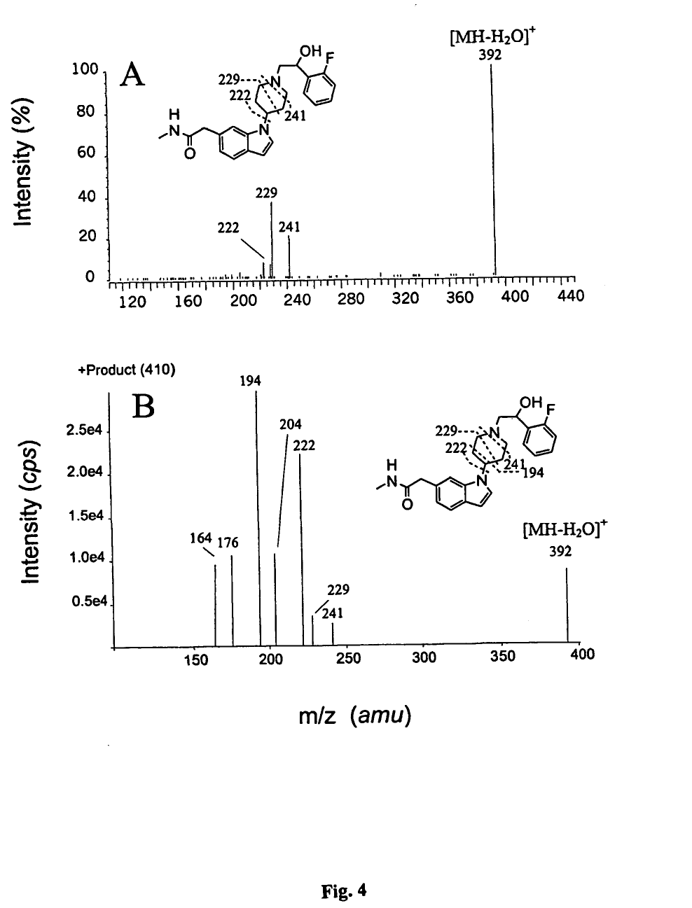

M1, one of the most abundant microsomal metabolites, was dissociated under CID in the triple quadrupole or the ion trip MS to the product ions at m/z 194, 222, 229, 241, and 392 (FIG. 4). These characteristic product ions suggested that the metabolite was Compound 1 hydroxylated at the fluorophenethyl piperidine moiety. The product ions of the metabolite at m/z 194 and 222 were likely the counterparts of Compound 1 at m/z 178 and 206, respectively. The product ions at m/z 176 and 164, however, were possibly secondary. In addition to the possible formation by the elimination of H₂O from the product ion at m/z 194, the ion at m/z 176 might be also produced by the sequential elimination of H₂O (m/z 204) and CH₂CH₂ from the product ion at m/z 222, while the ion at m/z 164 would be generated if such possible loss from the ion at m/z 222 was in addition to H₂O and CH₂CHCH₃, instead of CH₂CH₂. The abundant ion at m/z 392, apparently formed by the H₂O elimination from the MH⁺ ion, would further suggest the potential existence of an aliphatic, rather than an aromatic, hydroxyl group. Therefore, it would be reasonable to assign this metabolite as monohydroxylated Compound 1 with the hydroxyl group at one of the two carbons between the piperidinyl and the fluorophenyl group, preferably at the p-carbon to the piperidinyl nitrogen.

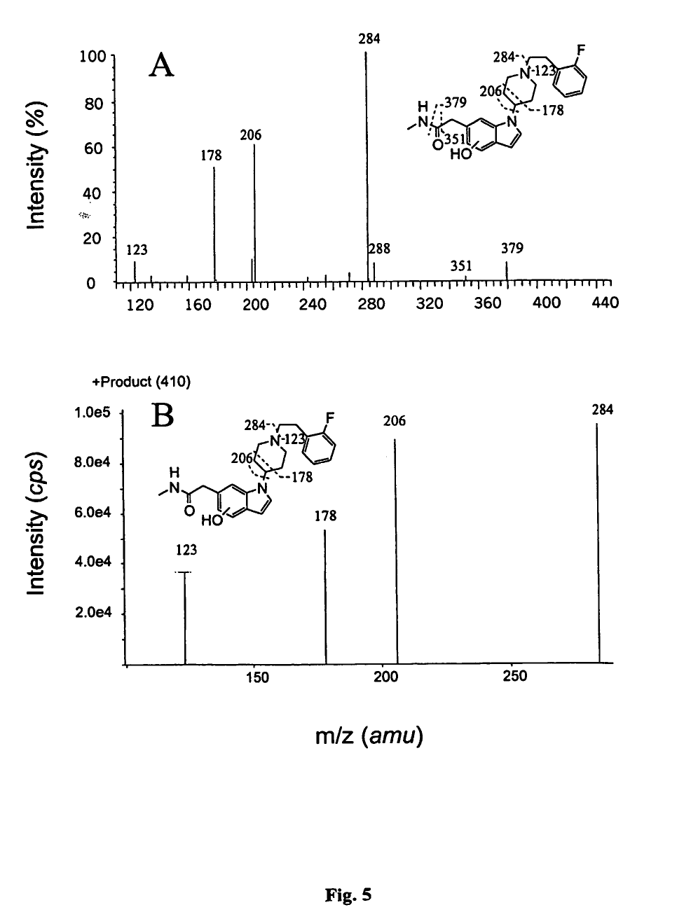

In contrast, the other major hydroxylated metabolite (M2) produced MS/MS product ions at m/z 206, 178, and 123 that are identical to those of Compound 1 (FIG. 5B). Therefore, the hydroxyl group of the metabolite was not likely at the fluorophenethyl piperidinyl moiety, which was further supported by the MS/MS spectrum produced by the ion trap mass spectrometer (FIG. 5A). The formation of product ions at m/z 351 and 379 in the ion trap MS indicated that the site of the hydroxyl group is in the indolyl acetamide, likely either on the indolyl ring or the carbon between the indolyl and amido group. However, the actual position of the M2 hydroxyl group could not be clarified. The lack of the MS/MS product ion at m/z 392 implied that the hydroxyl group was aromatic, rather than aliphatic. It is possible the multiple monooxidation at different sites, such as dihydroxylation of Compound 1.

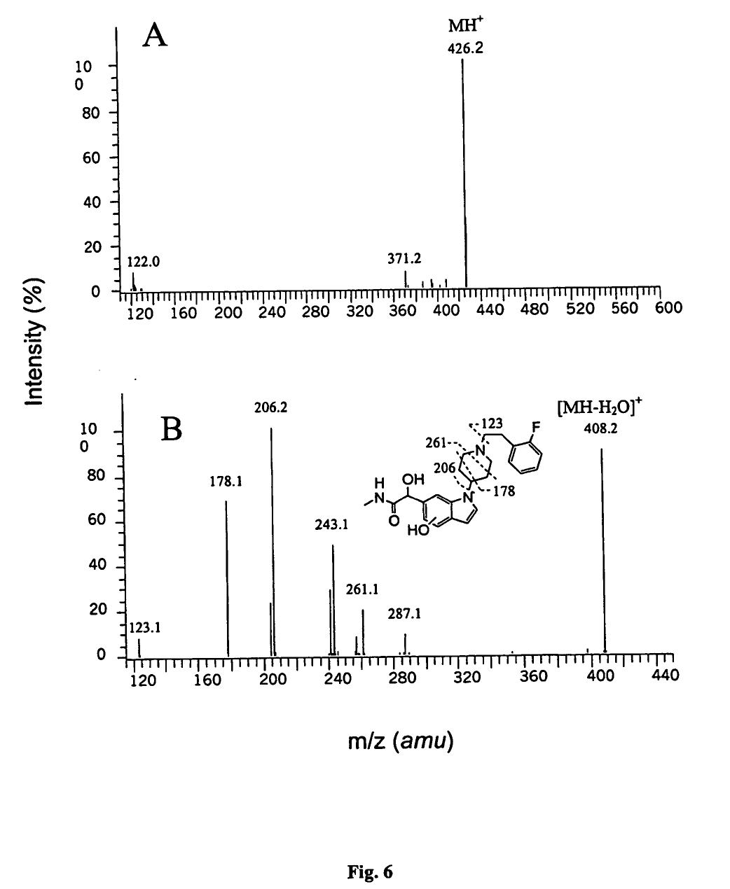

M3 was one of the major, if not the only, diol metabolite detected evidenced by the increment of 32 amu (m/z 426), as compared to that of Compound 1 (FIG. 6A). M3, similar to M2 and Compound 1, produced the MS/MS product ions at m/z 206, 178, and 123, and thus possessed the intact Compound 1 fluorophenethyl piperidinyl moiety (FIG. 6B). Interestingly, the contrast between the abundant product ion at m/z 408 and the lack of the product ion at m/z 390 indicated the possible elimination of one, but not likely two, H₂O molecules from the MH⁺ ion of M3 during the CID fragmentations, thus suggesting the possible co-existence of both aliphatic and aromatic hydroxyl group. Therefore, the sites where the hydroxyl groups attached would likely be in the indolyl ring between the indolyl and amido group.

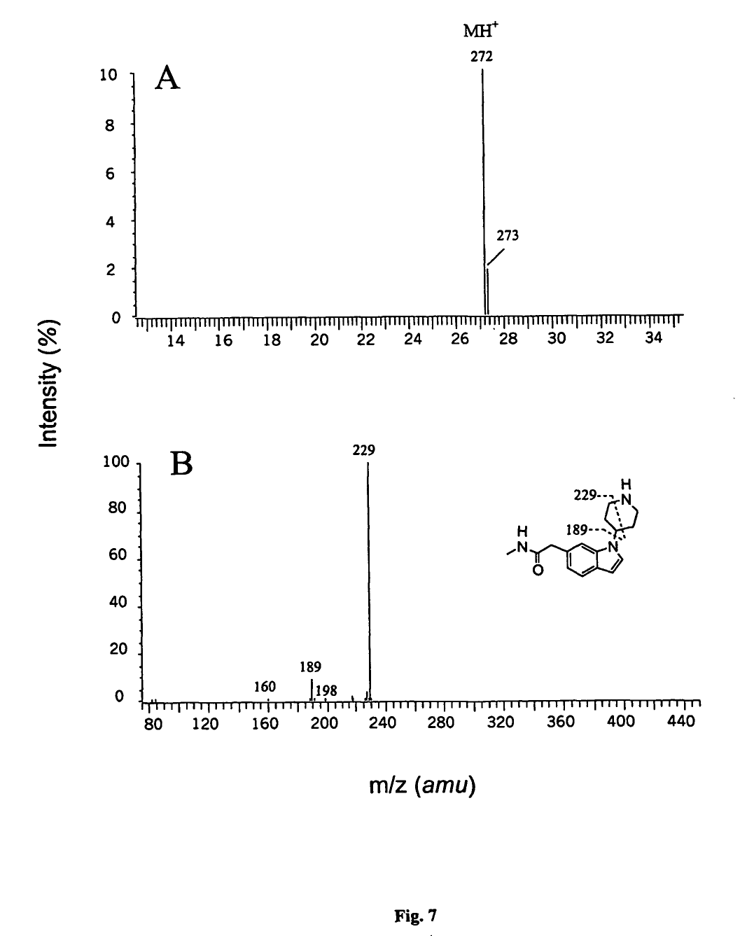

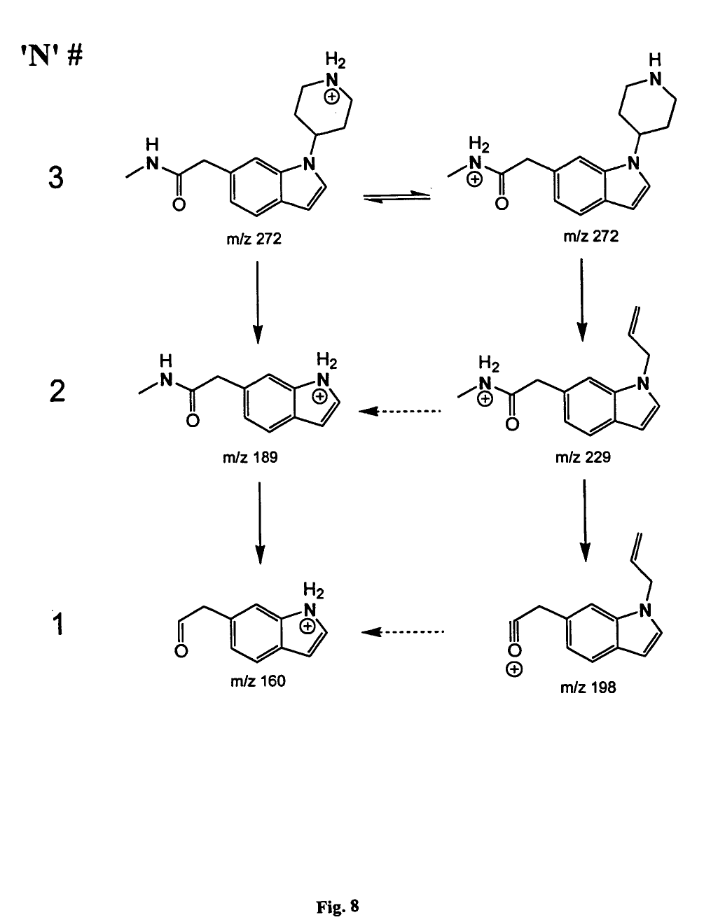

An N-dealkylated metabolite of Compound 1, in addition, was also detected. The structural elucidation of the dealkylated metabolite was straightforward, particularly when the nitrogen rule was applied (McLafferty et al., Interpretation of Mass Spectra, 4^{th} Edition, University Science Book, Sausalito, 1993). The MH⁺ ion at m/z 272 (FIG. 7A) indicated that M4 was a cleavage product of Compound 1, likely a dealkylated metabolite. The even m/z number of the MH⁺ ion of M4 suggested that the metabolite possessed an odd number of nitrogen atoms, as was the case for the MH⁺ ion of Compound 1 (FIG. 2). Therefore, the metabolite should have either one or three nitrogen atoms. Such a requirement would be fulfilled only if M4 was formed by the dealkylation at the piperidinyl nitrogen (FIG. 7B and FIG. 8).

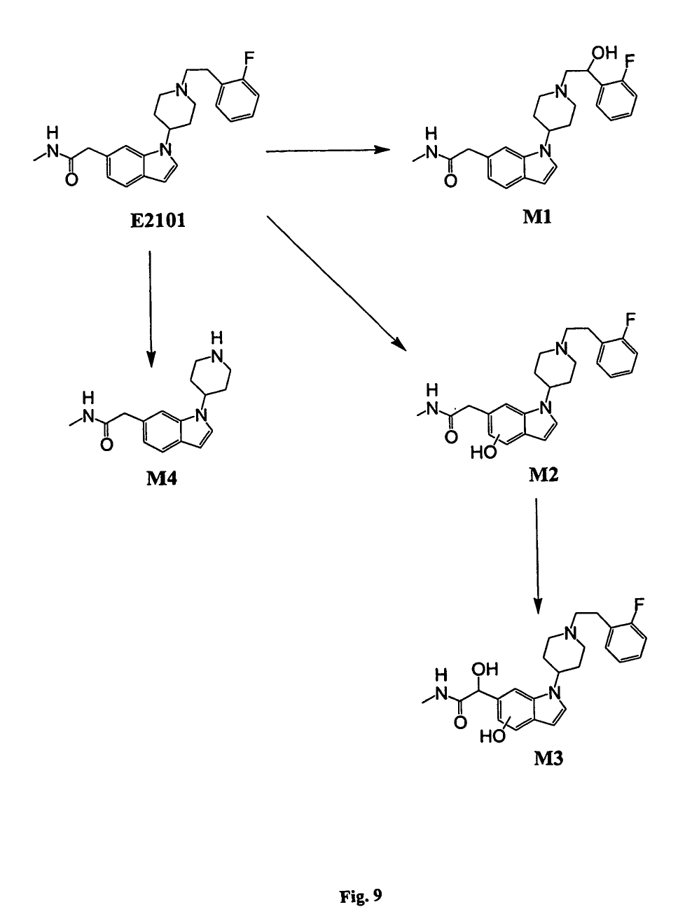

The major metabolic pathways of Compound 1 are proposed in FIG. 9.

Metabolic Enzyme Identification

The metabolic enzymes were identified by detecting the formation of metabolites in reconstituted enzyme systems. The rate of NADPH-dependent metabolism was found to be faster in the reaction mixture containing pooled HLM than that containing pooled HLS9 (FIG. 1). Therefore, the major metabolic enzymes of Compound 1 would be microsomal oxidases, or hepatic CYPs.

The responsible CYP forms for Compound 1 metabolism were further determined using recombinant human CYPs, including CYP1A2, CYP2C9, CYP2C19, CYP2D6, CYP2E1, and CYP3A4. In the presence of approximately 50 μM of Compound 1, CYP2C19, CYP2D6, and CYP3A4 metabolized Compound 1 (FIG. 1). The formation of the major Compound 1 metabolites was CYP form-dependent (Table 1). CYP3A4 produced the broadest spectrum of metabolites, similar to that generated by HLM or HLS9 preparations. However, CYP2C19 and CYP2D6 produced rather distinctive metabolite profiles. CYP2D6 preferably converted Compound 1 to M3 and to M2 to a much less extent, whereas CYP2C19 converted Compound 1 to all the major metabolites but M3. Apparently, the formation of the hydroxylated metabolite M2 was not metabolic CYP form-specific.

CYP Inhibition

A panel of CYP substrate assays was applied to determine the CYP form-specific inhibition as described in Experimental Methods. The quantifications were based on standard calibration curves. The correlation coefficient (r₂) for each calibration curve was at least 0.990. The analytical quality was also ensured by quality control samples. As is shown in Table 2, no inhibitory effect of Compound 1 at 10 or 50 μM on CYP1A2, CYP2C9, CYP2E1, or CYP3A4 activity was detected as assessed by R-warfarin 6(CYP1A2) and 10-hydroxylation (CYP3A4), S-warfarin 7-hydroxylation (CYP2C9), and chlorzoxazone 6-hydroxylation (CYP2E1). However, the activities of CYP2C 19-mediated S-mephenyloin 4′-hydroxylation and CYP2D6-mediated bufuralol 1′-hydroxylation were significantly reduced in the presence of Compound 1 in a concentration-dependent manner.

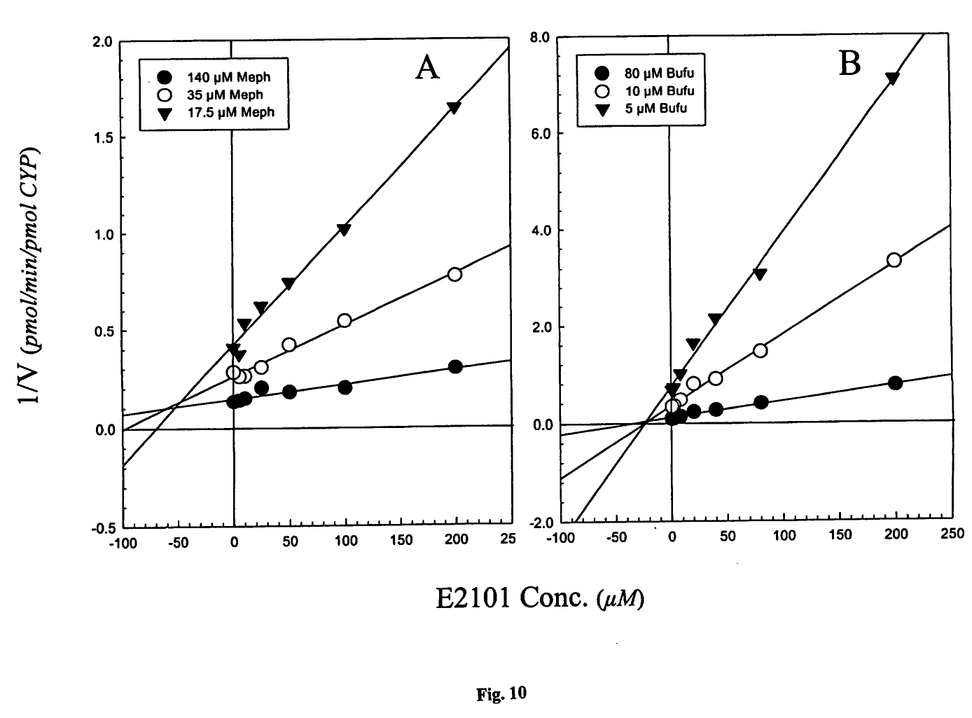

The apparent inhibition constants (Kᵢ) of CYP2C19 and CYP2D6 were determined using the microsomal preparations containing cDNA-expressed recombinant CYP proteins. The apparent K; values were first estimated by the Dixon plots. The estimated Kᵢ varied between 25-45 μM for CYP2C19 (FIG. 10A), and approximately 20 μM for CYP2D6 (FIG. 10B). The apparent Kᵢ values were also estimated by simultaneous nonlinear regression (SNLR) analysis using the common reversible inhibition models of Michaelis-Menten kinetics, including the competitive, uncompetitive, noncompetitive, and mixed inhibition model. The competitive inhibition model was selected to determine the Kᵢ based on the regression correlation coefficients (r²>(0.97 for CYP2C19 inhibition; r²>0.99 for CYP2D6 inhibition). In consistence with the Dixon plots, the apparent Kᵢ values determined by SNLR were 48 μM for CYP2C19 inhibition, and 15 μM for CYP2D6 inhibition. Both the Dixon plots and SNLR analyses suggested that the inhibitions of CYP2C19 and 2D6 by Compound 1 were primarily, if not fully, competitive.

CYP Induction

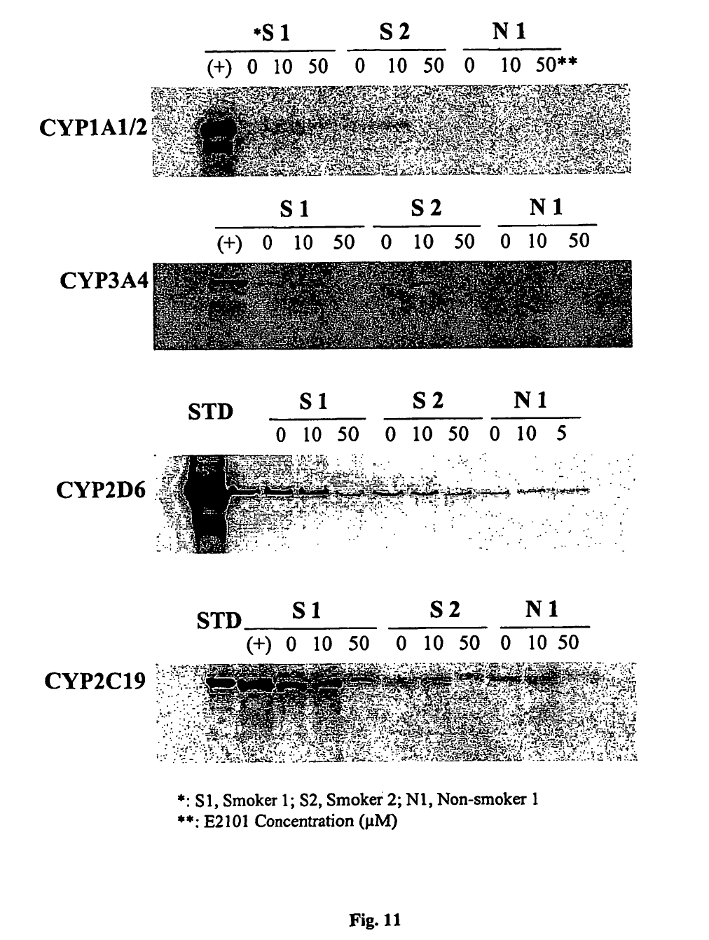

CYP induction was evaluated using primary culture of human hepatocytes from three donors (two smokers and one non-smoker). At the time the hepatocytes were received, the cells from the non-smoker were slightly more dense than those from the smokers. Though marked morphologic changes were not observed among the control cells and those exposed to Compound 1, the cell density tended to be reduced slightly during the treatment. With the exception of the monoclonal anti-CYP2D6 antibodies, the polyclonal primary antibodies used for immunochemical detection cross-reacted with the most of CYP subfamily members. Therefore, the enzymes determined were in fact CYP1Al/2, CYP2C8/9/19, CYP2D6, and CYP3A4/5. As shown in FIG. 11, the cells responded to inductions of CYP1A1/2, CYP3A4/5, and CYP2C19, as demonstrated by the elevated expressions of these proteins in the hepatocytes exposed to TCDD and rifampicin. However, the expression of CYP1A1/2, CYP2C19/2C8/2C9, CYP2D6, or CYP3A4/5 did not increase in the cells exposed to Compound 1 at 10 or 50 μM for 72 hours. Therefore, Compound 1 did not appear to induce the expression of these CYP forms.

TABLE 1
Relative Amount of Major Metabolites Formed in Reconstituted
Reaction Systems Containing HLM and Recombinant Human CYPs

| | Rel. Amount (%)^{a} | | | | | |
|---|---|---|---|---|---|---|
| | Major Metabolite | | | | | Compound |
| Enzyme | M1 | M2 | M3 | M4 | M1-4 | 1 |
| HLM | 11 | 3 | 3 | 21 | 38 | 36 |
| CYP2C19 | 30 | 7 | ND | 20 | 57 | 42 |
| CYP2D6 | ND | 2 | 8 | ND | 10 | 78 |
| CYP3A4 | 11 | 6 | 7 | 31 | 55 | 23 |

^{a}Estimated by the UV absorbance at 270 nm

TABLE 2
CYP Inhibition Profile of Compound 1 in Reconstituted Reaction System
Containing HLM

| Compound | | | | | | |
|---|---|---|---|---|---|---|
| 1 | Metabolic Rate (Mean ± SD; pmol/min/mg Protein)^{a,b} | | | | | |
| (μM) | CYP1A2 | CYP2C9 | CYP2C19 | CYP2D6 | CYP2E1 | CYP3A4 |
| 0 | 4.9 ± 0.7 | 0.81 ± | 49 ± 5 | 22 ± 0.9 | 193 ± 45 | 9.4 ± 4.3 |
| 10 | 4.3 ± 0.1 | 0.86 ± 0.00 | 44 ± 3 | 7.4 ± 2.5** | 142 ± 25 | 9.7 ± 1.7 |
| 50 | 4.4 ± 0.4 | 0.91 ± 0.05 | 33 ± 4* | 4.2 ± 0.4** | 185 ± 27 | 12.5 ± 1.1 |

^{a}Determined by CYP form-specific activities, including CYP1A2-mediated R-warfarin 6-hydroxylation, CYP2C9-mediated S-warfarin 7-hydroxylation, CYP2C19-mediated S-mephenytoin 4′-hydroxylation, CYP2D6-mediated bufuralol 1′-hydroxylation, CYP2E1-mediated chlorzoxazone 6-hydroxylation, and CYP3A4-mediated R-warfarin 10-hydroxylation.
^{b}N = 4.
*Significant difference from the controls (0.01 < p < 0.05).
**Highly significant difference from the controls (p < 0.01).

### Other Embodiments

All publications and patents mentioned herein are hereby incorporated by reference.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

Other embodiments are within the following claims.

1. A compound of the formula: 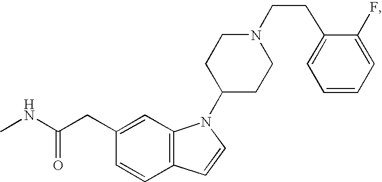
wherein one or more methylene or methine proton is replaced with a hydroxyl group.

2. The compound of claim 1, wherein said compound is of the formula: 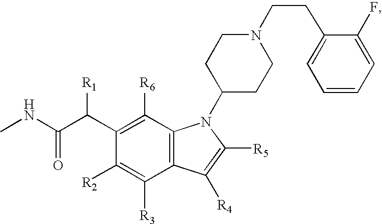
wherein R₁, R₂, R₃, R₄, R₅, and R₆ are each independently a hydrogen atom or a hydroxyl group, provided that at least one of R₁, R₂, R₃, R₄, R₅, and R₆ is a hydroxyl group.

3. The compound of claim 2, wherein only one of R₂, R₃, and R₆ is a hydroxyl group, and R₁, R₄, and R₅ are each hydrogen atoms.

4. The compound of claim 2, wherein only one of R₂, R₃, and R₆ is a hydroxyl group, R₁ is a hydroxyl group, and R₄ and R₅ are each hydrogen atoms.

5. The compound of claim 1, wherein said compound is of the formula: 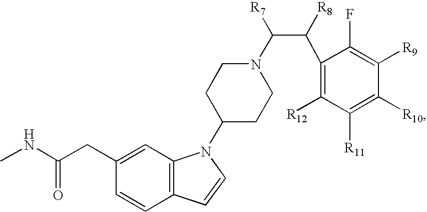
wherein R₇, R₈, R₉, R₁₀, R₁₁, and R₁₂ are each independently a hydrogen atom or a hydroxyl group, provided that at least one of R₇, R₈, R₉, R₁₀, R₁₁, and R₁₂ is a hydroxyl group.

6. The compound of claim 5, wherein only one of R₇, R₈, R₉, R₁₀, R₁₁, and R₁₂ is a hydroxyl group.

7. The compound of claim 6, wherein R₈ is a hydroxyl group.

8. A compound of the formula:

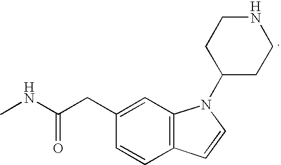

9. A pharmaceutical composition comprising the compound of claim 1 or claim 4 and a pharmaceutically acceptable carrier or diluent.

10. A method of preventing or treating a serotonergic disease or condition in a subject, said method comprising administering to the subject the pharmaceutical composition of claim 9.

11. Use of the compound of claim 1 or claim 4 in the prevention or treatment of a serotonergic disease or condition in a subject.

12. The method of claim 10 or claim 11, wherein said subject is a human.
